(19) United States
(12) Patent Application Publication
(10) Pub. No.: US 2009/0142412 A1
(43) Pub. Date: Jun. 4, 2009

(54) Skin Care Cosmetic Cream/Scrub Product

(76) Inventors: Giovanna Minutolo Cicillini, Pequannock, NJ (US); Diego A. Guillen, Haledon, NJ (US)

Correspondence Address: MYERS WOLIN, LLC, 100 HEADQUARTERS PLAZA, North Tower, 6th Floor, MORRISTOWN, NJ, 07960-6834, US

(21) Appl. No.: 12/327,805

(22) Filed: Dec. 3, 2008

Related U.S. Application Data
(60) Provisional application No. 60/992,096, filed on Dec. 3, 2007.

(51) Int. Cl. A61K 8/36; A61K 36/74; A61K 33/30; A61K 31/355; A61K 36/886; A61K 36/889; A61Q 19/00

(52) U.S. Cl. 424/642; 514/558; 424/725; 514/458; 424/744; 424/727

(57) ABSTRACT

A series of cosmetic scrubs and creams, some containing coffee, have the ability to moisturize, soften, scrub and revitalize body parts, create natural tanning qualities, and sooth itch from burns and bug bites.

### CROSS REFERENCE TO RELATED APPLICATION

The present application is a non provisional of U.S. application Ser. No. 60/992,096, filed on Dec. 3, 2007, pending, the specifications of which are in incorporated herein by reference.

### BACKGROUND OF THE INVENTION

Fonolla Moreno et al., US Pub. 20060134234, entitled Cosmetic composition discloses a composition containing, preferably in a physiologically acceptable medium, at least one xanthine base or a plant extract containing it, at least one polyurethane powder, and at least one non-ionic dimethicone copolyol. Where the xanthine base may be chosen from a plant extracts of tea, coffee, guarana, mate or cola.

Marty, U.S. Pat. No. 4,702,913, issued Oct. 27, 1987 entitled Novel cosmetic compositions discloses compositions that can also contain caffeine or theophylline or any product likely to contain them, such as tea or coffee.

Watkinson, U.S. Pat. No. 5,545,402, issued Aug. 13, 1996 entitled Skin care method and composition discloses the use of glycosidases include neuraminidase, mannosidase, galactosidase, glucosidase, N-acetyl glucosaminidase and N-acetyl galactosaminidase which are preferably isolated from plant sources including almonds, green coffee beans, and spinach.

Lanzendorfer, et al., U.S. Pat. No. 6,423,747, issued Jul. 23, 2002, entitled Cosmetic and dermatological preparations with flavonoids discloses the benefit of utilizing derivatives of cinnamic acid such as caffeic acid, which is contained, for example, in coffee, tobacco, poppy and dandelion.

Golz-Berner, et al., U.S. Pat. No. 6,989,150, issued Jan. 24, 2006 and entitled Cosmetic preparation of active substances with a synergistically increased radical protection factor discloses a cosmetic preparation of active substances, consisting of a bark extract of quebracho blanco which contains at least 90 wt. % of proanthocyanidine oligomers, a silkworm extract which contains the peptide cecropine, amino acids and a vitamin mixture, a non-ionic, cationic or anionic hydro-gel, phopholipids, a yeast disintegration product and cyclodextrines. The disclosed preparation may also contain additional active substances such as plant extracts of acerola, sea weed, citrus, bitter orange, cherry, papaya, tea, coffee beans, skin tree and angelica.

It is an object to create novel the coffee creams and scrubs product.

The product is recommended for young adults from age fifteen and older.

### SUMMARY OF THE INVENTION

The cosmetic scrub/cream comprises surfactant-thickening agents, emulsifier-thickening agents, emollients, stearic acid, distilled water, coffee grounds and an optional preservative.

### DETAILED DESCRIPTION

The novel skin care cosmetic cream/scrub product comprises one or more surfactant-thickening agents, one or more emulsifiers, emollients, stearic acid, ground coffee and water. It may optionally also contain a preservative.

The proportions of the various ingredients may vary depending on the identity of the specific components and the use of the product.

White petrolatum (Petroleum jelly), also known as petrolatum or soft paraffin is a semi-solid mixture of hydrocarbons. It is recognized by the U.S. Food and Drug Administration as an approved over-the-counter skin protectant and remains widely used in cosmetic skin care.

Petrolatum is a flammable, semi-solid mixture of hydrocarbons, having a melting-point usually ranging from a little below to a few degrees above 100° F. (37° C.). It is colorless or pale yellow, translucent, and devoid of taste and smell when pure. It does not oxidize on exposure to the air, and is not readily acted on by chemical reagents. It is insoluble in water.

In general, the petrolatum is present in an amount of from 0.25 to 17.5 percent by volume, preferably from about 0.5 to about 15 percent by volume, most preferably from about 1 to about 12 percent by volume.

Zinc oxide is used in some formulations for its ability to treat minor skin irritations (cuts, burns, and scrapes, poison ivy) by providing a skin barrier to prevent and help heal skin irritation.

In general, if present, the zinc oxide is present in an amount of from about 1 to about 4 percent by volume.

In general the surfactants can be present in an amount of from about 0.5 to about 10 percent by volume, preferably from about 1 to about 5 percent by volume, most preferably from about 1.5 to about 4 percent by volume.

A preferred surfactant is sodium lauryl sulfate.

Cetyl alcohol is a nonionic surfactant emollient, emulsifier and thickener.

Cetyl alcohol (1-hexadecanol) can be present in an amount of from about 0.5 to about 12.5 percent by volume, preferably from about 1 to about 10 percent by volume, most preferably from about 1 to about 4 percent by volume.

Stearic acid (octadecanoic acid) is present in the formulation and can be present in an amount of from about 0.5 to about 7.5 percent by volume, preferably from about 1 to about 5 percent by volume, most preferably from about 1.5 to about 4 percent by volume.

The stearic acid can be present in an amount of from about 0.5 to about 7.5 percent by volume, preferably from about 1 to about 6 percent by volume, most preferably from about 1.5 to about 4 percent by volume.

The emollient can be any of the commonly used in cosmetics. Liquid glycerin is a preferred emollient.

The emollient can be present in an amount of from about 1 to about 15 percent by volume, preferably from about 1 to about 13 percent by volume, most preferably from about 2.5 to about 11 percent by volume.

Coffee or coffee ground may be present in the formulation. The coffee is brewed coffee, preferably brewed espresso coffee. When present, the coffee can be present in an amount of from about 25 to about 65 percent by volume, preferably from about 30 to about 60 percent by volume, most preferably from about 40 to about 55 percent by volume.

When present, the coffee grounds may be used or fresh, and the grind may be of any convenient size. It is preferred to utilize used fully extracted finely ground coffee grounds.

The coffee grounds can be present in an amount of from about 1 to about 10 percent by volume, preferably from about 2 to about 8 percent by volume, most preferably from about 4 to about 7.5 percent by volume.

Coconut oil is excellent when used as a skin moisturizer and softener and is as effective and safe as mineral oil when used as a moisturizer, with an absence of adverse reactions.

The coconut oil, if optionally present, can be present in an amount of from about 10 to about 60 percent by volume, preferably from about 15 to about 50 percent by volume, most preferably from about 20 to about 30 percent by volume.

The water can be present in an amount of from about 40 to about 90 percent by volume, preferably from about 60 to about 90 percent by volume, most preferably from about 65 to about 85 percent by volume.

Fragrances may optionally be added. Preferred fragrances are cappuccino hazelnut, pear glace or fructis fragrances, coconut lime verbena, and mango mandarin.

When present, fragrances are present in amounts of from about 0.4 to 2 percent by volume.

Colorants may optionally be added to the formulation. Preferred colorants are FD&C Yellow #5, FD&C Red #3 and FD&C Green #5.

When present, colorants are present in amount of from about 0.4 to 2 percent by volume.

Real fruit or plant material may optionally be added to the formulation to provide color, texture and fragrance. Preferred materials are mango, melon and aloe. Fruit is preferably added in combination with matching or complementary fragrances and colorants to provide layers of color, texture and fragrance to the formulation.

When present, the plant material is present in amounts of from 15 to 30 percent.

Fruit or plant juices are optionally included in the formulations. If present, the juices are present in an amount of from 1.5 to 4 percent by volume.

Vitamin E, α-tocopherol, is an important lipid-soluble antioxidant, that it protects cell membranes from oxidation by removing free radical intermediates and preventing the oxidation reaction from continuing. The oxidised α-tocopheroxyl radicals produced in this process are recycled back to the active reduced form through reduction by other antioxidants, such as retinol.

The Vitamin E can be present in an amount of from about 0.05 to about 0.15 percent by volume.

Vitamin A can be found in various forms retinol. It functions, in conjunction with Vitamin E, as an antioxidant in the formulation.

The Vitamin A can be present in an amount of from about 0.05 to about 0.15 percent by volume.

The optional preservative is present in a minor amount. The preservative can be any of the commonly used in cosmetics. Liquapar is a preferred preservative. It is effective against bacteria, yeast and molds.

The following non-limiting examples further illustrate the invention.

### EXAMPLE 1

A preparation for diminishing skin blemishes is prepared from the following ingredients
200 ml distilled water
15 ml cooked (extracted) coffee grounds
10 ml glycerin
5 ml cetyl alcohol
5 ml stearic acid
5 ml sodium lauryl sulfate
0.04 ml white petrolatum
1.6 ml cappuccino hazelnut
0.4 ml liquapar optima
   by melting the petrolatum in hot water and adding the alcohol, stearic acid, sodium lauryl sulfate, glycerin and heat to below boiling. The extracted coffee grinds are added and the formulation stirred while heating to boiling. After boiling for approximately 1 minute remove from heat, stir for approximately 20-seconds, and add the cappuccino hazelnut and liquapar optima. Cool to room temperature.

This formulation exfoliates the skin, softens the skin and lasts for days, promotes tanning the skin if in the sun, helps remove acne from faces in a few days, and may be used as a shaving cream. It leaves the skin feeling clean and soft.

### EXAMPLE 2

The composition of example 1 is applied to the skin before shaving and is used as and replaces shaving cream.

### EXAMPLE 3

The composition of example 1 is applied with light rubbing motion to the skin, and allowed to remain on the skin for 3 minutes or more before washing it off.

### EXAMPLE 4

A preparation for diminishing the itch caused by bug-bites (mosquitoes, bees, etc.) and for reducing the swelling, redness and rash and minimizing the symptoms of poison ivy is prepared from the following ingredients
200 ml distilled water
30 ml glycerin
15 ml cetyl alcohol
5 ml stearic acid
5 ml sodium lauryl sulfate
5 ml white petrolatum
15 ml parsley extract
5 ml zinc oxide
1.6 ml pear glace or fructis fragrances
0.4 ml liquapar optima
   by melting the petrolatum in hot water with the zinc oxide and adding the cetyl alcohol, stearic acid, sodium lauryl sulfate, glycerin and heating to boiling. After boiling for approximately 1 minute remove from heat, stir for approximately 20 seconds, and add the pear glace or fructis fragrances and liquapar optima. Cool to room temperature.

### EXAMPLE 5

To diminish the itch caused by bug-bites (mosquitoes, bees, etc.) and for reducing the swelling, redness and rash and minimizing the symptoms of poison ivy the formulation of Example 4 is applied to the affected parts of the body by gently rubbing the formulation onto the skin.

### EXAMPLE 6

A preparation for softening the skin is prepared from the following ingredients
100 ml distilled water
45 ml coconut oil
5 ml glycerin
5 ml cetyl alcohol
5 ml stearic acid
5 ml sodium lauryl sulfate
10 ml white petrolatum
0.4 ml liquapar optima
1.2 ml coconut lime verbena
0.16 ml Vitamin E Oil
0.16 ml Vitamin A
   by melting the petrolatum in hot water and adding the cetyl alcohol, stearic acid, sodium lauryl sulfate, and glycerin and heating to boiling. After boiling for approximately 1 minute remove from heat, stir for approximately 1 minute, and add the vitamin E, vitamin A, coconut lime verbena, and liquapar optima. Cool to room temperature.

### EXAMPLE 7

The formulation of Example 7 is applied to skin to be softened and/or moisturized and left on the skin.

### EXAMPLE 8

A preparation for cleansing the skin and removing make-up is prepared from the following ingredients
100 ml distilled water
45 ml coconut oil
5 ml glycerin
5 ml cetyl alcohol
5 ml stearic acid
5 ml sodium lauryl sulfate
15 ml white petrolatum
0.4 ml liquapar optima
35 ml fresh melon
5 ml Mango Pure
1.2 ml Mango Mandarin
0.4 ml Fruit Extract
0.4 ml Liquapar Optima
0.16 ml Vitamin E
0.08 ml FD&C Yellow #5
0.08 ml FD&C Red #3
   by melting the petrolatum in hot water and adding the cetyl alcohol, stearic acid, sodium lauryl sulfate, melon, mango pure and glycerin and heating to boiling. After boiling for approximately 1 minute remove from heat, stir for approximately 1 minute, and add the fruit extract, vitamin E, yellow and red coloring, and liquapar optima. Cool to room temperature.

### EXAMPLE 9

To soften and cleanse the skin the formulation of Example 8 is applied as a mask by gently rubbing onto the skin and allowed to remain on the skin for at least 1 minute. It is then washed off.

### EXAMPLE 10

A preparation for minimizing wrinkles on the skin is prepared from the following ingredients
100 ml distilled water
45 ml natural (fresh) aloe vera
10 ml glycerin
5 ml cetyl alcohol
5 ml stearic acid
5 ml sodium lauryl sulfate
15 ml white petrolatum
0.4 ml liquapar optima
5 ml natural aloe vera juice concentrate
1.2 ml green clover & aloe
0.16 ml Vitamin E oil
0.16 ml Vitamin A
0.08 ml FD&C Yellow #5
0.08 ml FD&C Green #5
   by melting the petrolatum in hot water and adding the cetyl alcohol, stearic acid, sodium lauryl sulfate, and glycerin and heating to boiling. Add aloe vera and aloe vera juice. After boiling for approximately 1 minute remove from heat, stir for approximately 1 minute, and add the vitamin E, vitamin A, food coloring, liquapar and green clover and aloe. Cool to room temperature.

### EXAMPLE 11

To minimize wrinkles, skin burns, sunburns, and rashes on the skin the formulation of Example 10 is applied to affected portions of the body and allowed to remain on the skin.

### EXAMPLE 12

A preparation for preventing the eruption of acne and eczema and giving the skin a natural shine is prepared from the following ingredients
100 ml distilled water
100 ml espresso coffee
10 ml glycerin
5 ml sodium lauryl sulfate
5 ml stearic acid
5 ml cetyl alcohol
2.5 ml white petrolatum
0.20 ml cappuccino hazelnut
0.20 ml liquapar optima
   by melting the petrolatum in hot water and adding the cetyl alcohol, stearic acid, sodium lauryl sulfate, and glycerin and heating to boiling. After boiling for approximately 1 minute remove from heat, stir for approximately 1 minute, and add the liquapar and cappuccino hazelnut. Cool to room temperature.

### EXAMPLE 13

The formulation of Example 12 is rubbed gently onto the skin. The espresso coffee combined with the other ingredients minimizes the eruption of acne and eczema and it gives the skin a natural shine.

1. A skin care cosmetic comprising water, glycerin, cetyl alcohol, white petrolatum, stearic acid, liquapar optima and a surfactant.

2. The skin care cosmetic of claim 1 also containing espresso coffee.

3. The skin care cosmetic of claim 1 also containing extracted coffee grounds.

4. The skin care cosmetic of claim 1 further comprising a fragrance.

5. The skin care cosmetic of claim 1 further comprising a colorant.

6. The skin care cosmetic of claim 1 further comprising zinc oxide.

7. The skin care cosmetic of claim 1 further comprising vitamin A and vitamin E.

8. The skin care cosmetic of claim 1 further comprising fresh fruit.

9. The skin care cosmetic of claim 1 further comprising fruit extracts.

10. The skin care cosmetic of claim 1 further comprising aloe.

11. The skin care cosmetic of claim 1 for diminishing the itch caused by bug-bites (mosquitoes, bees, etc.) and for reducing the swelling, redness and rash and minimizing the symptoms of poison ivy comprising
distilled water
glycerin
cetyl alcohol
stearic acid
sodium lauryl sulfate
white petrolatum
parsley extract
zinc oxide
pear glace or fructis fragrances
liquapar optima

12. The skin care cosmetic of claim 1 for diminishing the itch caused by bug-bites (mosquitoes, bees, etc.) and for reducing the swelling, redness and rash and minimizing the symptoms of poison ivy comprising
200 ml distilled water
30 ml glycerin
15 ml cetyl alcohol
5 ml stearic acid
5 ml sodium lauryl sulfate
5 ml white petrolatum
15 ml parsley extract
5 ml zinc oxide
1.6 ml pear glace or fructis fragrances
0.4 ml liquapar optima

13. The skin care cosmetic of claim 1 for minimizing wrinkles on the skin comprising
distilled water
natural (fresh) aloe vera
glycerin
cetyl alcohol
stearic acid
sodium lauryl sulfate
white petrolatum
liquapar optima
natural aloe vera juice concentrate
green clover & aloe
Vitamin E oil
Vitamin A
FD&C Yellow #5
FD&C Green #5

14. The skin care cosmetic of claim 1 for minimizing wrinkles on the skin comprising
100 ml distilled water
45 ml natural (fresh) aloe vera
10 ml glycerin
5 ml cetyl alcohol
5 ml stearic acid
5 ml sodium lauryl sulfate
15 ml white petrolatum
0.4 ml Liquapar Optima
5 ml natural aloe vera juice concentrate
1.2 ml green clover & aloe
0.16 ml Vitamin E oil
0.16 ml Vitamin A
0.08 ml FD&C Yellow #5
0.08 ml FD&C Green #5

15. The skin care cosmetic of claim 1 for diminishing skin blemishes comprising
distilled water
extracted coffee grounds
glycerin
cetyl alcohol
stearic acid
sodium lauryl sulfate
white petrolatum
cappuccino hazelnut
liquapar optima

16. The skin care cosmetic of claim 1 for diminishing skin blemishes comprising
200 ml distilled water
15 ml extracted coffee grounds
10 ml glycerin
5 ml cetyl alcohol
5 ml stearic acid
5 ml sodium lauryl sulfate
2.5 ml white petrolatum
1.6 ml cappuccino hazelnut
0.4 ml liquapar optima

17. The skin care cosmetic of claim 1 for softening the skin comprising
distilled water
coconut oil
glycerin
cetyl alcohol
stearic acid
sodium lauryl sulfate
white petrolatum
liquapar optima
coconut lime verbena
Vitamin E Oil
Vitamin A

18. The skin care cosmetic of claim 1 for softening the skin comprising
100 ml distilled water
45 ml coconut oil
5 ml glycerin
5 ml cetyl alcohol
5 ml stearic acid
5 ml sodium lauryl sulfate
10 ml white petrolatum
0.4 ml liquapar optima
1.2 ml coconut lime verbena
0.16 ml Vitamin E Oil
0.16 ml Vitamin A

19. The skin care cosmetic of claim 1 for cleansing the skin and removing make-up is prepared from the following ingredients
distilled water
coconut oil
glycerin
cetyl alcohol
stearic acid
sodium lauryl sulfate
white petrolatum
liquapar optima
fresh melon
Mango Pure
Mango Mandarin
Fruit Extract
Liquapar Optima
Vitamin E
FD&C Yellow #5
FD&C Red #3

20. The skin care cosmetic of claim 1 for cleansing the skin and removing make-up is prepared from the following ingredients
100 ml distilled water
45 ml coconut oil
5 ml glycerin
5 ml cetyl alcohol
5 ml stearic acid
5 ml sodium lauryl sulfate
15 ml white petrolatum
0.4 ml liquapar optima
35 ml fresh melon
5 ml Mango Pure
1.2 ml Mango Mandarin
0.4 ml Fruit Extract
0.4 ml Liquapar Optima
0.16 ml Vitamin E
0.08 ml FD&C Yellow #5
0.08 ml FD&C Red #3
